(19) United States
(12) Patent Application Publication
(10) Pub. No.: US 2024/0148767 A1
(43) Pub. Date: May 9, 2024

(54) THE USE OF EMPAGLIFLOZIN FOR THE TREATMENT OF ULCERATIVE COLITIS AND CROHN'S DISEASE

(71) Applicant: The Governors of the University of Alberta, Edmonton (CA)

(72) Inventors: Jason DYCK, Edmonton (CA); Karen Lynn MADSEN, St. Albert (CA)

(21) Appl. No.: 18/279,517

(22) PCT Filed: Mar. 3, 2022
(86) PCT No.: PCT/CA2022/050297

§ 371 Date: Aug. 30, 2023

Related U.S. Application Data
(60) Provisional application No. 63/156,537, filed on Mar. 4, 2021.

(51) Int. Cl. A61K 31/7048; A61P 1/04

(52) U.S. Cl. CPC A61K 31/7048; A61P 1/04

(57) ABSTRACT

A use of empagliflozin for the treatment of ulcerative colitis and Crohn's disease.

### CROSS REFERENCE TO RELATED APPLICATION

This application claims priority to U.S. provisional patent application 63/156,537, filed Mar. 4, 2021, the entire contents of which is hereby incorporate by reference.

### FIELD

The present disclosure relates generally to the use of empagliflozin for the treatment of ulcerative colitis and Crohn's disease.

### BACKGROUND

Inflammatory bowel diseases (IBD), including Crohn's disease (CD) and ulcerative colitis (UC) are characterized by sustained intestinal mucosa inflammation, caused mainly by excessive macrophage activation and inflammatory T effector cells. Although significant advances have been made in recent years in the treatment of IBD using immunomodulators, a large percentage of patients do not respond to current available therapies and of those who do initially respond, a large number lose responses to therapy over time. In addition, current IBD mediations are associated with significant infectious and possible neoplastic side effects. Thus, development of new therapies with improved safety profiles are required.

The concept of immunometabolism has emerged recently whereby repolarizing of inflammatory immune cells towards anti-inflammatory profiles by manipulating cellular metabolism represents a new therapeutic approach¹. As effector and regulatory immune cell populations have different metabolic requirements, this allows for cellular selectivity when regulating immune responses based on metabolic pathways. Unlike the approach of global immunosuppression, targeting specific metabolic processes can selectively target cells with high metabolic demands while not affecting other immune cells thus reducing potential side effects. These effects could include reprogramming classically activated M1 macrophages towards a more anti-inflammatory M2 macrophage and/or repolarizing circulating T cell effector subsets towards a more anti-inflammatory Treg population¹.

SGLT2 inhibitors are effective in controlling blood glucose levels in patients with Type 2 diabetes Empagliflozin (EMPA) is a sodium-glucose co-transporter-2 (SGLT2) inhibitor. EMPA acts to inhibit glucose reabsorption in the proximal tubule of the kidney

### SUMMARY

In one aspect, there is provided a method of treating a subject with ulcerative colitis (UC), comprising administering a therapeutically effective amount of Empagliflozin (EMPA).

In one aspect, there is provided a method of treating a subject with Crohn's disease (CD), comprising: administering a therapeutically effective amount of Empagliflozin (EMPA).

In one example, the subject is a human.

In one aspect, there is provided a use of effective amount of Empagliflozin (EMPA) for treating a subject with ulcerative colitis (UC).

In one aspect, there is provided a use of effective amount of Empagliflozin (EMPA) in the manufacture of a medicament for treating a subject with ulcerative colitis (UC).

In one aspect, there is provided a use of effective amount of Empagliflozin (EMPA) for treating a subject with Crohn's disease (CD).

In one aspect, there is provided a use of effective amount of Empagliflozin (EMPA) in the manufacture of a medicament for treating a subject with Crohn's disease (CD).

In one example, the subject is a human.

In one aspect, there is provided a kit for treating a subject with ulcerative colitis (UC), comprising: Empagliflozin (EMPA), a container, and optionally instructions for the use thereof.

In one aspect, there is provided a kit for treating a subject with Crohn's disease (CD), comprising: Empagliflozin (EMPA), a container, and optionally instructions for the use thereof.

### BRIEF DESCRIPTION OF THE FIGURES

Embodiments of the present disclosure will now be described, by way of example only, with reference to the attached Figures.

FIG. 1A-B depicts improved weight gain. (A) Colonic Weight/Length Ratio. (B) Weight difference from day 0 to day 14.

FIG. 2A-B depicts EMPA treatment decreased stool lipocalin levels and improved colonic histological scores. (A) Stool Lipocalin-2. (B) Total score.

FIG. 3A-D. Adult IL-10−/− mice with established colitis were treated with EMPA (10 mg/kg) daily via gavage for 14 days. EMPA treated mice showed reduction in stool lipocalin-2, maintained their weight, had reduced colonic weight/length, and showed complete enterocyte healing as evidenced by no enterocyte injury. This was associated with reduced neutrophilic and lymphocytic infiltration into the lamina propria. (A) depicts Enterocytes injury. (B) depicts Lamina Propria (Neutrophils). (C) depicts Lamina Propria (Lymphocytes). (D) depicts Epithelial hyperplasia.

FIG. 4. Representative histological samples of colons. Colons were flushed with phosphate-buffered saline containing 0.1% gentamycin and immediately fixed in 10% v/v neutral buffered formalin. The fixed samples were paraffin-embedded, sectioned, and stained with hematoxylin and eosin. Disease scoring methods used were based on four scores: 1) epithelial hyperplasia (0-3), 2) enterocyte injury (0-3), 3) lymphocytes (0-2) and neutrophil infiltration (0-2) in the lamina propria. The total maximum histological score (10) is based on the sum of the individual scores. (A) control. (B) Empa.

FIG. 5A-D. Effect of EMPA treatment on colonic cytokines. EMPA treated mice showed significant reduction in colonic expression of IFNγ, IL-1β, and TNFα . . . (A) depicts INFγ Colon (rt-PCT). (B) depicts IL1β colon (rt-PCT). (C) depicts IL6 Colon (rt-PCR). (D) depicts TNFα Colon (rt-PCR).

### DETAILED DESCRIPTION

In one aspect, there is provided a method of treating a subject with ulcerative colitis (UC), comprising: administering a therapeutically effective amount of Empagliflozin (EMPA).

In one aspect, there is provided a method of treating a subject with Crohn's disease (CD), comprising: administering a therapeutically effective amount of Empagliflozin (EMPA).

The term “Crohn's disease” refers to a type of inflammatory bowel disease characterized by inflammation of the lining of the gastrointestinal tract. Symptoms may include diarrhea, abdominal pain, fever, fatigue, bloody stool and weight loss.

The term “active” Crohn's disease as used herein refers to Crohn's disease that is biologically active. Patients with active disease may be symptomatic and exhibit one or more sign or symptom of Crohn's disease for example, abdominal pain, increased stool frequency, mucosal inflammation or abnormal laboratory tests (e.g., elevated ESR or CRP values or decreased hemoglobin or increased faecal calprotectin). “Refractory” Crohn's disease with respect to a particular therapy refers to Crohn's disease that is active or that relapses or flares in spite of being treated with that therapy. Chronically active disease refers to disease that requires continuous treatment for relief of symptoms.

“Ulcerative colitis” is normally continuous from the rectum up the colon. The disease is classified by the extent of involvement, depending on how far up the colon the disease extends, into (a) distal colitis, which includes proctitis, proctosigmoiditis and left-sided colitis, and (b) extensive colitis, which includes pancolitis.

The term “ulcerative colitis” as used herein refers to any one of the forms in which the disease presents itself.

The term “active” ulcerative colitis as used herein refers to ulcerative colitis that is biologically active. Patients with active disease may be symptomatic and exhibit one or more sign or symptom of ulcerative colitis, for example, rectal bleeding, increased stool frequency, mucosal inflammation or abnormal laboratory tests (e.g., elevated ESR or CRP values or decreased hemoglobin or increased faecal calprotectin). “Refractory” ulcerative colitis with respect to a particular therapy refers ulcerative colitis that is active or that relapses or flares in spite of being treated with that therapy. Chronic ulcerative colitis refers to a disease characterized by a chronic inflammation of the rectal and colonic mucosa.

The term “inflammatory bowel disease” refers to a pathology characterized by an inflammatory condition of the colon and/or the small intestine. Crohn's disease and colitis are two types of inflammatory bowel disease.

Empagliflozin (EMPA) is a highly selective sodium glucose cotransporter-2 (SGLT2) inhibitor which is effective in the treatment of individuals with type 2 diabetes.² Interestingly, it has been demonstrated in human trials that EMPA treatment exerts potent cardioprotective effects independent of glycemic control but involving cardiac inflammation. Further, EMPA has also been shown to suppress LPS-induced renal and systemic inflammation in a mouse model.³

We undertook studies to determine if EMPA treatment may also be effective in reducing gut inflammation.

SGLT2 inhibitors: SGLT2 inhibitors (canagliflozin, dapagliflozin, empagliflozin, ertugliflozin) were originally designed as kidney-targeting hypoglycemic drugs used to manage type 2 diabetes⁴ but have since been shown to possess multiple pharmacological relevant protective effects, including anti-apoptotic, anti-inflammatory and antioxidant effects^{4, 5}. In the kidney, glucose is filtered by the renal glomeruli and then reabsorbed in the proximal convoluted tubule through the actions of facilitated glucose transporters (GLUTs) and active sodium-glucose cotransporters (SGLT1 and SGLT2). In diabetic patients, SGLT2 inhibitors work by inhibiting glucose reabsorption through SGLT2 and facilitating its excretion in urine leading to decreases in plasma glucose levels. SGLT2 is primarily expressed in the kidney but mRNA for SGLT2 is also expressed in small amounts in other tissues in the body, including T cells and macrophages⁶⁻⁸. Interestingly, several of the effects of SGLT2 inhibitors are seen in tissues with no apparent SGLT2 expression, suggesting that many of the described beneficial effects of these drugs may be mediated by SGLT2-independent mechanisms which are yet to be determined. Of the four SGLT2 inhibitors in clinical use, Empaglifozin (EMPA: Jardiance, Boehringer Ingelheim) has the highest selectivity for SGLT2 over SGLT1 (˜2700 fold); thus, we have chosen to focus our studies on EMPA². EMPA has a bioavailability of 78% and is rapidly absorbed in the small intestine reaching peak levels 1.5 hours after a single dose with a half-life of ˜12 hours in humans. Approximately 40% of EMPA can be recovered in the feces as unchanged drug indicating that direct exposure of the colon to the drug occurs⁹.

Clinical Effects of SGLT2 inhibitors: Although SGLT2 inhibitors were originally designed for the management of diabetes, these drugs have rapidly expanded into other therapeutic areas including being used for protection from heart failure, chronic kidney disease, non-alcoholic fatty liver disease, type 1 diabetes, obesity, and gout⁵. This interest in the use of SGLT2 for these other indications occurred due to the unexpected findings in several clinical trials in diabetic patients that the use of SGLT2 inhibitors had profound reno- and cardio-protective effects in the absence of any effect on plasma glucose levels. These findings subsequently led to trials investigating the benefits of SGLT2 inhibitors in non-diabetic patients for the treatment of heart failure and chronic kidney disease. To date, three large clinical trials have confirmed beneficial effects of SGLT2 inhibitors in non-diabetic patients with chronic kidney disease and in patients with heart failure¹⁰⁻¹². EMPA treatment has also demonstrated improvement of liver steatosis and fibrosis in patients with NAFLD and type 2 diabetes^{13, 14}. As SGLT2 is not expressed in either heart or liver, the protective mechanisms underlying how these effects of EMPA are mediated remain to be determined.

Effects of SGLT2 inhibitors on inflammation: Substantial evidence supports an anti-inflammatory role for SGLT2 inhibitors through both direct and indirect effects on inflammatory signaling and oxidative stress pathways resulting in altered cytokine production in a variety of different cell types⁵. SGLT2 inhibitors have been shown to suppress macrophage infiltration into heart, liver, and kidneys in animal models of insulin resistance and diabetes and to polarize macrophages towards an M2 phenotype¹⁵⁻¹⁹. In an obese murine model, EMPA attenuated inflammation and fibrosis by suppressing T cells and cytotoxic T lymphocytes^{19, 20}. Recent work has identified beneficial cardiac effects of EMPA to be associated with an EMPA-induced decrease in cardiac inflammation through a direct inhibition of the NLRP3 inflammasome in macrophages and decreases in IL-1β and TNFα secretion²¹. The NLRs family member NLRP3 is rapidly emerging as a crucial regulator of intestinal homeostasis. This innate immune receptor mediates the assembly of the inflammasome complex in the presence of microbial ligands, triggering activation of caspase-1 and secretion of interleukin-1β(IL-1β) and IL-18 and has been implicated in the pathogenesis of IBD²²; thus therapy aimed at inhibition of NLRP3 may have great potential in the management of IBD.

AMPK and Inflammation: There is evidence that SGLT2 inhibitors exert these effects on NLRP3 through the activation of AMPK^{21, 23}. Adenosine monophosphate (AMP) activated protein kinase (AMPK), a serine/threonine kinase, is a central regulator of metabolism and energy balance in epithelial cells and lymphocytes²⁴. AMPK senses changes in cellular energy levels and activates pathways through a phosphorylation mechanism that generate ATP and inhibiting biological pathways that consume ATP consumption, thus leading to ATP production and energy restoration. AMPK is expressed in epithelial cells along the entire gastrointestinal tract²⁵. AMPK is reduced under chronic inflammatory conditions and activation of AMPK has been shown to enhance intestinal absorption, improve barrier function, and reduce gut inflammation^{24, 26}. Thus, AMPK activation through pharmacological intervention has the potential to be a promising new therapeutic strategy for treatment of inflammatory intestinal disorders. EMPA has been shown to exert anti-inflammatory effects through activation of the AMPK pathway in several cell types²⁷⁻²⁹. In animal models, EMPA treatment improves cardiac function due to an AMPK-mediated attenuation of oxidative stress, inhibition of cardiomyocyte apoptosis and maintenance of mitochondrial membrane potential integrity^{30, 31}. EMPA also has been shown to improve hepatic steatosis and improve NAFLD-related liver injury in mouse models through enhancing macrophage autophagy and inhibiting the IL-17/IL-23 axis through the AMPK pathway^{32, 33}.

Macrophages and IBD: Macrophages are key effector cells of the innate immune system and contribute to the pathogenesis of IBD³⁴. Macrophages orchestrate T-cell recruitment and activation as well as remodeling extracellular components in tissue which can contribute to granuloma and fibrosis in CD patients. During homeostasis, blood-derived monocytes enter the lamina propria where they differentiate into tolerogenic IL-10-producing macrophages that do not produce pro-inflammatory cytokines when challenged with commensal bacteria³⁴. However, in both CD and UC patients this process can be dysregulated with increased migration seen as well as differentiation of monocytes into pro-inflammatory macrophages that release large amounts of pro-inflammatory cytokines in response to microbial stimuli^{35, 36}. Further, macrophages in CD patients can display abnormal maturation with prolonged intracellular bacterial survival and have an enhanced ability to induce expansion of pathogenic Th17 cells³⁶. There is evidence for a causal association between defective resolution of gut inflammation and altered macrophage differentiation that results in impaired bacterial clearance and excessive cytokine secretion in patients with IBD³⁷⁻⁴⁰. A recent study has shown that peripheral blood monocyte-derived macrophages isolated from CD patients have impaired bacterial clearance along with reduced secretion of pro-inflammatory cytokines due to significant metabolic derangements⁴¹. In addition, while expression of key M1 genes involved in T-cell recruitment and activation were upregulated in macrophages from both UC and CD patients, macrophages from CD patients also expressed an M2 profile and increased granuloma and fibrosis phenotypes^{41, 42}.

There is conflicting information as to EMPA effects on macrophages with both positive and negative results being reported^{21, 23, 43, 44}. For example, in vitro studies in murine macrophages demonstrated that EMPA treatment attenuated LPS-induced TNFα and iNOS expression and increased expression of anti-inflammatory M2 markers through an AMPK-mediated mechanism. Further, EMPA also prevented LPS-induced ATP depletion and altered phenotype and activity of macrophages⁴⁴. In human THP-1 macrophages, EMPA pretreatment for 24 hours decreased TNFα-stimulated chemokine release⁴⁶. Macrophages isolated from diabetic patients treated with EMPA had reduced NLRP3 activation and IL-1β expression but whether this was a direct effect of EMPA or an indirect due to EMPA-induced changes in host metabolism was not determined²³. To date, effects of EMPA treatment on macrophages from IBD patients have not been examined. If EMPA treatment could be demonstrated to improve metabolic function and alter macrophage phenotypes through pharmacological modulation of intracellular signaling pathways, this could be a novel and powerful approach to treat gut inflammation.

T cells and IBD: There is substantial evidence implicating T cells and T-cell migration to the gut in initiating and perpetuating gut inflammation in patients with IBD⁴⁶. A dysregulated and excessive T cell response is seen in IBD patients with active inflammation with increased CD4+T effector cells, T regulatory cells and lower numbers of CD8+ T cells and CD103+ T cells^{47, 48}. Intestinal inflammation in IBD patients has generally been attributed to CD4+ subsets; however, autoreactive CD8+ cells have also been suggested to have a role in the initiation of inflammation due to their damaging actions on barrier function allowing luminal microbes access to the lamina propria; this increased exposure would subsequently attract and expand CD4+T effector cells⁴⁷. Increased levels of Tregs are often seen in CD patients with active disease; this has been suggested to represent active recruitment of these cells to suppress inflammation, which possibly fails due to either too few cells, impaired function, or conversion of Treg cells to Th17 cells within the lamina propria⁴⁹. Drug therapies aimed at targeting T cells such as thiopurines which induce T cell apoptosis; vedolizumab, which blocks T-cell trafficking through blocking the α4 β7 integrin/MAdCAM-1 interaction; and anti-IL-12/23p40 which interferes with Th1 and Th17 lymphocytes are effective in treating subgroups of IBD patients⁴⁶.

AMPK and T cells: AMPK is activated in T cells by both immune signals and environmental stimuli and plays an important role in T cell metabolism. AMPK functions in T cells to modulate cellular proliferation and differentiation, memory T cell development and cytokine production⁵⁰. Upon encountering antigen and co-stimulatory signals from antigen presenting cells, naïve T cells switch to a program of anabolic growth to promote expansion of antigen-specific T cells. This switch from quiescent to proliferative state requires increased ATP. Triggering AMPK during this period can influence T cell differentiation towards Treg cells rather than T effector subsets⁵⁰. These findings support the potential for therapeutics targeting AMPK activation to be an effective strategy for modulating T cell phenotypic differentiation from pathogenic effector subtypes to regulatory subsets in IBD patients.

Effects of SGLT2 Inhibitors in Animal Models of Colitis and Preliminary Data: Studies using the SGLT2 inhibitor, dapagliflozin, in the 2,4,6 trinitrobenzene sulfonic acid (TNBS)-induced rat colitis model demonstrated beneficial effects of dapagliflozin against experimental colitis via augmenting colonic autophagy and curbing apoptosis through activation of AMPK and suppression of HMGB1/RAGE/NF-κcascade⁵¹. This study showed preventative effect rather than treatment of existing inflammation.

The term “subject”, as used herein, refers to an animal, and can include, for example, domesticated animals, such as cats, dogs, etc., livestock (e.g., cattle, horses, pigs, sheep, goats, etc.), laboratory animals (e.g., mouse, rabbit, rat, guinea pig, etc.), mammals, non-human mammals, primates, non-human primates, rodents, birds, reptiles, amphibians, fish, and any other animal. In a specific example, the subject is a human.

The term “treatment”, “treat”, or “treating” as used herein, refers to obtaining beneficial or desired results, including clinical results. Beneficial or desired clinical results can include, but are not limited to, alleviation or amelioration of one or more symptoms or conditions, diminishment of extent of disease, stabilized (i.e. not worsening) state of disease, preventing spread of disease, delay or slowing of disease progression, amelioration or palliation of the disease state, diminishment of the reoccurrence of disease, and remission (whether partial or total), whether detectable or undetectable. “Treating” and “Treatment” can also mean prolonging survival as compared to expected survival if not receiving treatment.

In one example, “remission” in a subject or patient suffering from Crohn's disease refers to when their CDAI score is <150. In one example, “remission” in a subject or patient suffering from Ulcerative Colitis refers to when their total Mayo score is 0, when their total Mayo score is less than or equal to 2, or when their total Mayo score is less than or equal to 2 with no category score above 1.

The term “amelioration” or “ameliorates” as used herein refers to a decrease, reduction or elimination of a condition, disease, disorder, or phenotype, including an abnormality or symptom.

The term “symptom” of a disease or disorder (e.g., ulcerative colitis or Crohn's disease) is any morbid phenomenon or departure from the normal in structure, function, or sensation, experienced by a subject and indicative of disease.

In another example, a subject with ulcerative colitis or Crohn's disease can be treated to provide cellular or biological responses, a complete response, a partial response, a stable disease (without progression or relapse), or a response with a later relapse of the patient from or as a result of the treatment.

A compound or composition may be administered alone or in combination with other treatments, either simultaneously or sequentially, dependent upon the condition to be treated.

In treating a subject, a therapeutically effective amount may be administered to the subject.

As used herein, the term “therapeutically effective amount” refers to an amount that is effective for preventing, ameliorating, or treating a disease or disorder (e.g., ulcerative colitis or Crohn's disease).

The formulations may conveniently be presented in unit dosage form and may be prepared by any methods well known in the art of pharmacy. Such methods include the step of bringing the active compound into association with a carrier, which may constitute one or more accessory ingredients. In general, the formulations are prepared by uniformly and intimately bringing into association the active compound with liquid carriers or finely divided solid carriers or both, and then if necessary shaping the product.

The compounds and compositions may be administered to a subject by any convenient route of administration, whether systemically/peripherally or at the site of desired action, including but not limited to, oral (e.g. by ingestion); topical (including e.g. transdermal, intranasal, ocular, buccal, and sublingual); pulmonary (e.g. by inhalation or insufflation therapy using, e.g. an aerosol, e.g. through mouth or nose); rectal; vaginal; parenteral, for example, by injection, including subcutaneous, intradermal, intramuscular, intravenous, intraarterial, intracardiac, intrathecal, intraspinal, intracapsular, subcapsular, intraorbital, intraperitoneal, intratracheal, subcuticular, intraarticular, subarachnoid, and intrasternal; by implant of a depot/for example, subcutaneously or intramuscularly.

Compounds and/or compositions comprising compounds disclosed herein may be used in the methods described herein in combination with standard treatment regimes, as would be known to the skilled worker.

The therapeutic formulation may also contain more than one active compound as necessary for the particular indication being treated, typically those with complementary activities that do not adversely affect each other. Such molecules are suitably present in combination in amounts that are effective for the purpose intended.

A skilled worked will be able to determine the appropriate dose for the individual subject by following the instructions on the label. Preparation and dosing schedules for commercially available second therapeutic and other compounds administered in combination with or concomitantly with compounds or compositions described herein may be used according to manufacturers' instructions or determined empirically by the skilled practitioner.

Aim: The aim of this study was to examine the effects of treatment with EMPA on colitis in a mouse model of inflammatory bowel disease and to determine if effects are due to a direct interaction.

Methods: In our colony, IL-10−/− mice begin to develop colitis between 8-12 weeks of age. Adult IL-10−/− mice with demonstrated colitis as evidenced by stool lipocalin-2 values (>20 pg/g)⁵² were started on EMPA (10 mg/kg daily gavage) or vehicle. This dose of EMPA was initially chosen based on studies in animal models showing beneficial effects^{3, 21, 53, 54}. Further, this dose was also chosen to match the active dose in humans by considering the differences in the metabolism and other pharmacokinetic parameters between mice and humans⁵⁵. The dose used in this study results in free plasma concentration of ˜20 nmol/L in mice⁵⁶ which is within the normal and safe range of that reported in humans⁵⁷⁻⁵⁹. Further, this dose does not cause hypoglycemia in healthy humans and had no effect on blood glucose levels in our pilot experiments. Adult IL-10^{-/-} mice with established colitis were treated with a daily gavage of EMPA (10 mg/kg; n=10) or vehicle (n=10) for 14 days. Disease activity was assessed by measurement of mouse weight, colonic weight and length, histological score, cytokine levels in colonic homogenate and lipocalin-2 levels in stool. To examine for possible direct effects of EMPA, colonic explants from wild-type and IL-10^{-/-} mice were incubated with increasing doses of EMPA (0.1-5 μM)±LPS for 2 hours and tissue levels of IL-1β and TNFα measured.

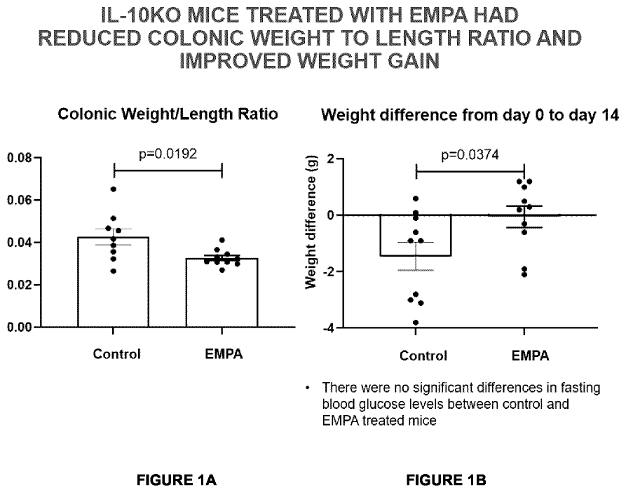

FIG. 1A-B depicts improved weight gain. (A) Colonic Weight/Length Ratio. (B) Weight difference from day 0 to day 14.

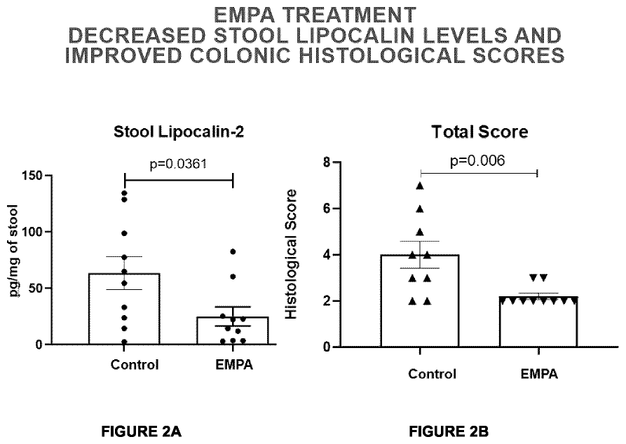

FIG. 2A-B depicts EMPA treatment decreased stool lipocalin levels and improved colonic histological scores. (A) Stool Lipocalin-2. (B) Total score.

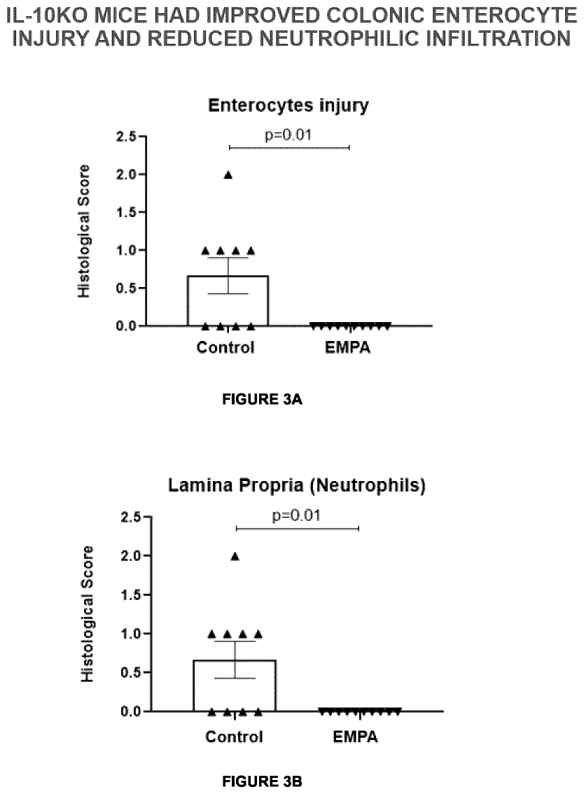

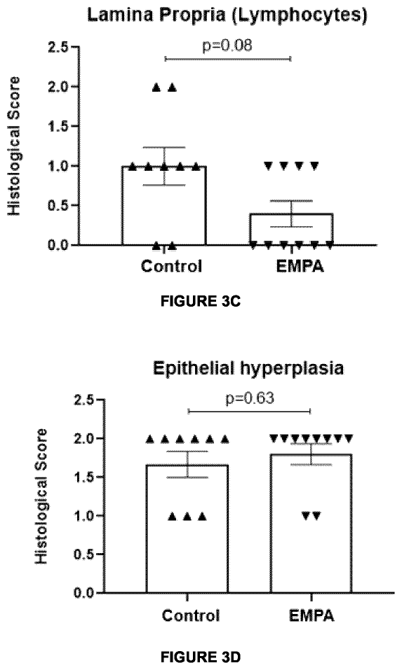

FIG. 3A-D. Adult IL-10−/− mice with established colitis were treated with EMPA (10 mg/kg) daily via gavage for 14 days. EMPA treated mice showed reduction in stool lipocalin-2, maintained their weight, had reduced colonic weight/length, and showed complete enterocyte healing as evidenced by no enterocyte injury. This was associated with reduced neutrophilic and lymphocytic infiltration into the lamina propria. (A) depicts Enterocytes injury. (B) depicts Lamina Propria (Neutrophils). (C) depicts Lamina Propria (Lymphocytes). (D) depicts Epithelial hyperplasia.

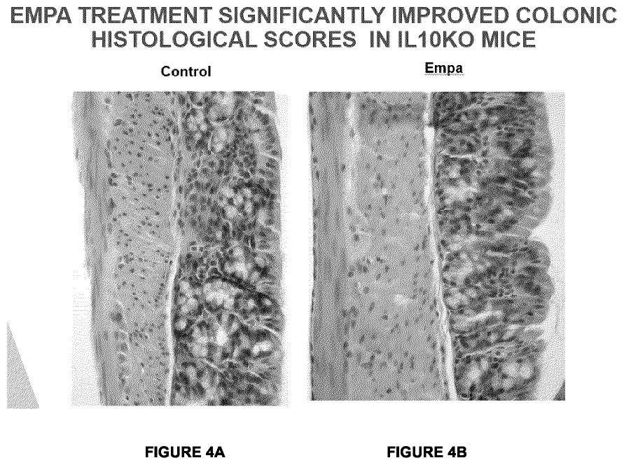

FIG. 4. Representative histological samples of colons. Colons were flushed with phosphate-buffered saline containing 0.1% gentamycin and immediately fixed in 10% v/v neutral buffered formalin. The fixed samples were paraffin-embedded, sectioned, and stained with hematoxylin and eosin. Disease scoring methods used were based on four scores: 1) epithelial hyperplasia (0-3), 2) enterocyte injury (0-3), 3) lymphocytes (0-2) and neutrophil infiltration (0-2) in the lamina propria. The total maximum histological score (10) is based on the sum of the individual scores. (A) control. (B) Empa.

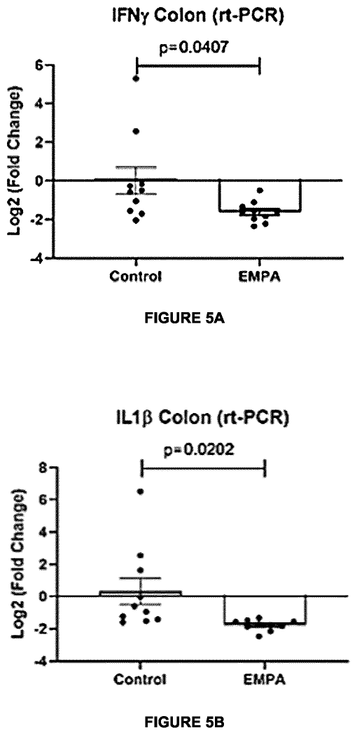

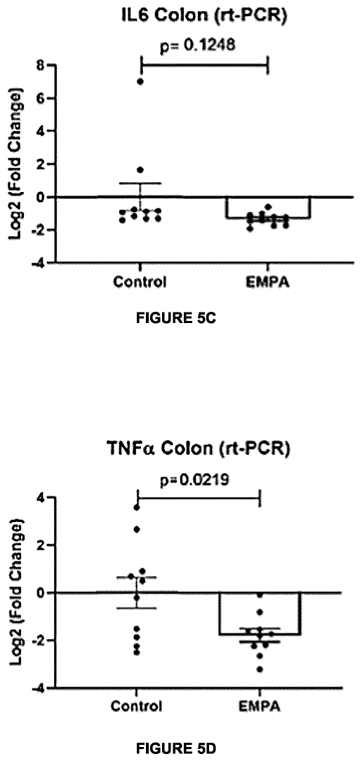

FIG. 5A-D. Effect of EMPA treatment on colonic cytokines. EMPA treated mice showed significant reduction in colonic expression of IFNγ, IL-1α, and TNFα.lymphocytic infiltration into the lamina propria. (A) depicts INFγ Colon (rt-PCT). (B) depicts IL1β colon (rt-PCT). (C) depicts IL6 Colon (rt-PCR). (D) depicts TNFα Colon (rt-PCR).

Results: After 14 days EMPA treated IL-10^{-/-} mice with established colitis had a significant improvement in colonic inflammation as evidenced by a decreased colonic weight to length ratio (p=0.019), a decrease in fecal lipocalin-2 (p=0.03) and a significant decrease in enterocyte injury (p<0.01) decreased lamina propria neutrophils(p=0.01) and decreased total histological score (p=0.006). EMPA treated mice also maintained their weight over the 14 days while vehicle treated mice continued to lose weight (p=0.04). There were no significant differences in blood glucose levels between EMPA-treated mice and controls. Real-time qPCR revealed significant decreases in colonic IFNy (p=0.04), IL-1β (p=0.04), and TNFα (p=0.02) in IL-10−/− mice treated with EMPA. There were no significant differences in microbial composition or diversity between groups or over time, suggesting that the EMPA-induced improvement in inflammation was not mediated through microbial changes.

Conclusion: EMPA treatment significantly improved inflammation and maintained body weight in adult IL-10−/− mice with established colitis.

### CONCLUSIONS

EMPA treatment significantly improved histologic and fecal and tissue inflammatory markers and maintained body weight in adult IL-10K0 mice with established colitis

The mechanisms underlying the beneficial effects of EMPA remain to be determined but the improvement seen in IL-10K° mice suggests the involvement of a non-IL-10 dependent mechanism

Method of the invention are conveniently practiced by providing the compounds and/or compositions used in such method in the form of a kit. Such kit preferably contains the composition. Such a kit preferably contains instructions for the use thereof.

The embodiments described herein are intended to be examples only. Alterations, modifications and variations can be effected to the particular embodiments by those of skill in the art. The scope of the claims should not be limited by the particular embodiments set forth herein, but should be construed in a manner consistent with the specification as a whole.

### REFERENCES

1. Palsson-McDermott E M, O'Neill L A J. Targeting immunometabolism as an anti-inflammatory strategy. Cell Res 2020; 30:300-314.
2. Grempler R, Thomas L, Eckhardt M, et al. Empagliflozin, a novel selective sodium glucose cotransporter-2 (SGLT-2) inhibitor: characterisation and comparison with other SGLT-2 inhibitors. Diabetes Obes Metab 2012; 14:83-90.
3. Maayah Z H, Ferdaoussi M, Takahara S, et al. Empagliflozin suppresses inflammation and protects against acute septic renal injury. Inflammopharmacology 2021; 29:269-279.
4. Tentolouris A, Vlachakis P, Tzeravini E, et al. SGLT2 Inhibitors: A Review of Their Antidiabetic and Cardioprotective Effects. Int J Environ Res Public Health 2019; 16.
5. Kalra S, Shetty K K, Nagarajan V B, et al. Basic and Clinical Pharmaco-Therapeutics of SGLT2 Inhibitors: A Contemporary Update. Diabetes Ther 2020; 11:813-833.
6. Uhlen M, Fagerberg L, Hallstrom B M, et al. Proteomics. Tissue-based map of the human proteome. Science 2015; 347:1260419.
7. Chen J, Williams S, Ho S, et al. Quantitative PCR tissue expression profiling of the human SGLT2 gene and related family members. Diabetes Ther 2010; 1:57-92.
8. Bhaysar S K, Singh Y, Sharma P, et al. Expression of JAK3 Sensitive Na+ Coupled Glucose Carrier SGLT1 in Activated Cytotoxic T Lymphocytes. Cell Physiol Biochem 2016; 39:1209-28.
9. Ndefo U A, Anidiobi N O, Basheer E, et al. Empagliflozin (Jardiance): A Novel SGLT2 Inhibitor for the Treatment of Type-2 Diabetes. P T 2015; 40:364-8.
10. Heerspink H J L, Stefansson B V, Correa-Rotter R, et al. Dapagliflozin in Patients with Chronic Kidney Disease. N Engl J Med 2020; 383:1436-1446.
11. McMurray J J V, Solomon S D, Inzucchi S E, et al. Dapagliflozin in Patients with Heart Failure and Reduced Ejection Fraction. N Engl J Med 2019; 381:1995-2008.
12. Packer M, Anker S D, Butler J, et al. Cardiovascular and Renal Outcomes with Empagliflozin in Heart Failure. N Engl J Med 2020; 383:1413-1424.
13. Chehrehgosha H, Sohrabi M R, Ismail-Beigi F, et al. Empagliflozin Improves Liver Steatosis and Fibrosis in Patients with Non-Alcoholic Fatty Liver Disease and Type 2 Diabetes: A Randomized, Double-Blind, Placebo-Controlled Clinical Trial. Diabetes Ther 2021; 12:843-861.
14. Scheen A J. Beneficial effects of SGLT2 inhibitors on fatty liver in type 2 diabetes: A common comorbidity associated with severe complications. Diabetes Metab 2019; 45:213-223.
15. Benetti E, Mastrocola R, Vitarelli G, et al. Empagliflozin Protects against Diet-Induced NLRP-3 Inflammasome Activation and Lipid Accumulation. J Pharmacol Exp Ther 2016; 359:45-53.
16. Chiazza F, Couturier-Maillard A, Benetti E, et al. Targeting the NLRP3 Inflammasome to Reduce Diet-Induced Metabolic Abnormalities in Mice. Mol Med 2016; 21:1025-1037.
17. Gembardt F, Bartaun C, Jarzebska N, et al. The SGLT2 inhibitor empagliflozin ameliorates early features of diabetic nephropathy in BTBR ob/ob type 2 diabetic mice with and without hypertension. Am J Physiol Renal Physiol 2014; 307:F317-25.
18. Tahara A, Kurosaki E, Yokono M, et al. Effects of sodium-glucose cotransporter 2 selective inhibitor ipragliflozin on hyperglycaemia, oxidative stress, inflammation and liver injury in streptozotocin-induced type 1 diabetic rats. J Pharm Pharmacol 2014; 66:975-87.
19. Xu L, Nagata N, Nagashimada M, et al. SGLT2 Inhibition by Empagliflozin Promotes Fat Utilization and Browning and Attenuates Inflammation and Insulin Resistance by Polarizing M2 Macrophages in Diet-induced Obese Mice. EBioMedicine 2017; 20:137-149.
20. Park H J, Han H, Oh E Y, et al. Empagliflozin and Dulaglutide are Effective against Obesity-induced Airway Hyperresponsiveness and Fibrosis in A Murine Model. Sci Rep 2019; 9:15601.
21. Byrne N J, Matsumura N, Maayah Z H, et al. Empagliflozin Blunts Worsening Cardiac Dysfunction Associated With Reduced NLRP3 (Nucleotide-Binding Domain-Like Receptor Protein 3) Inflammasome Activation in Heart Failure. Circ Heart Fail 2020; 13:e006277.
22. Zhen Y, Zhang H. NLRP3 Inflammasome and Inflammatory Bowel Disease. Front Immunol 2019; 10:276.
23. Kim S R, Lee S G, Kim S H, et al. SGLT2 inhibition modulates NLRP3 inflammasome activity via ketones and insulin in diabetes with cardiovascular disease. Nat Commun 2020; 11:2127.
24. Sun X, Zhu M J. AMP-activated protein kinase: a therapeutic target in intestinal diseases. Open Biol 2017; 7.
25. Sun X, Yang Q, Rogers C J, et al. AMPK improves gut epithelial differentiation and barrier function via regulating Cdx2 expression. Cell Death Differ 2017; 24:819-831.
26. O'Neill L A, Hardie D G. Metabolism of inflammation limited by AMPK and pseudo-starvation. Nature 2013; 493:346-55.
27. Lu Q, Li X, Liu J, et al. AMPK is associated with the beneficial effects of antidiabetic agents on cardiovascular diseases. Biosci Rep 2019; 39.
28. Hawley S A, Ford R J, Smith B K, et al. The Na+/Glucose Cotransporter Inhibitor Canagliflozin Activates AMPK by Inhibiting Mitochondrial Function and Increasing Cellular AMP Levels. Diabetes 2016; 65:2784-94.
29. Zhou H, Wang S, Zhu P, et al. Empagliflozin rescues diabetic myocardial microvascular injury via AMPK-mediated inhibition of mitochondrial fission. Redox Biol 2018; 15:335-346.
30. Liu Y, Wu M, Xu J, et al. Empagliflozin prevents from early cardiac injury post myocardial infarction in non-diabetic mice. Eur J Pharm Sci 2021:105788.
31. Andreadou I, Efentakis P, Balafas E, et al. Empagliflozin Limits Myocardial Infarction in Vivo and Cell Death in Vitro: Role of STAT3, Mitochondria, and Redox Aspects. Front Physiol 2017; 8:1077.
32. Li T, Fang T, Xu L, et al. Empagliflozin Alleviates Hepatic Steatosis by Activating the AMPK-TET2-Autophagy Pathway in vivo and in vitro. Front Pharmacol 2020; 11:622153.
33. Meng Z, Liu X, Li T, et al. The SGLT2 inhibitor empagliflozin negatively regulates IL-17/IL-23 axis-mediated inflammatory responses in T2D M with NAFLD via the AMPK/mTOR/autophagy pathway. Int Immunopharmacol 2021; 94:107492.
34. Na Y R, Stakenborg M, Seok S H, et al. Macrophages in intestinal inflammation and resolution: a potential therapeutic target in IBD. Nat Rev Gastroenterol Hepatol 2019; 16:531-543.
35. Bernardo D, Marin A C, Fernandez-Tome S, et al. Human intestinal pro-inflammatory CD11c(high)CCR2(+)CX3CR1(+) macrophages, but not their tolerogenic CD11c(−)CCR2(−)CX3CR1(−) counterparts, are expanded in inflammatory bowel disease. Mucosal Immunol 2018; 11:1114-1126.
36. Ogino T, Nishimura J, Barman S, et al. Increased Th17-inducing activity of CD14+CD163 low myeloid cells in intestinal lamina propria of patients with Crohn's disease. Gastroenterology 2013; 145:1380-91 el.
37. Schwerd T, Pandey S, Yang H T, et al. Impaired antibacterial autophagy links granulomatous intestinal inflammation in Niemann-Pick disease type Cl and XIAP deficiency with NOD2 variants in Crohn's disease. Gut 2017; 66:1060-1073.
38. Palmer C D, Rahman F Z, Sewell G W, et al. Diminished macrophage apoptosis and reactive oxygen species generation after phorbol ester stimulation in Crohn's disease. PLoS One 2009; 4:e7787.
39. Rahman F Z, Hayee B, Chee R, et al. Impaired macrophage function following bacterial stimulation in chronic granulomatous disease. Immunology 2009; 128:253-9.
40. Smith A M, Rahman F Z, Hayee B, et al. Disordered macrophage cytokine secretion underlies impaired acute inflammation and bacterial clearance in Crohn's disease. J Exp Med 2009; 206:1883-97.
41. Dharmasiri S, Garrido-Martin E M, Harris R J, et al. Human Intestinal Macrophages Are Involved in the Pathology of Both Ulcerative Colitis and Crohn Disease. Inflamm Bowel Dis 2021.
42. Xue J, Schmidt S V, Sander J, et al. Transcriptome-based network analysis reveals a spectrum model of human macrophage activation. Immunity 2014; 40:274-88.
43. Leng W, Ouyang X, Lei X, et al. The SGLT-2 Inhibitor Dapagliflozin Has a Therapeutic Effect on Atherosclerosis in Diabetic ApoE(−/−) Mice. Mediators Inflamm 2016; 2016:6305735.
44. Koyani C N, Plastira I, Sourij H, et al. Empagliflozin protects heart from inflammation and energy depletion via AMPK activation. Pharmacol Res 2020; 158:104870.
45. Ortega R, Collado A, Selles F, et al. SGLT-2 (Sodium-Glucose Cotransporter 2) Inhibition Reduces Ang I I (Angiotensin I I)-Induced Dissecting Abdominal Aortic Aneurysm in ApoE (Apolipoprotein E) Knockout Mice. Arterioscler Thromb Vasc Biol 2019; 39:1614-1628.
46. Ahluwalia B, Moraes L, Magnusson M K, et al. Immunopathogenesis of inflammatory bowel disease and mechanisms of biological therapies. Scand J Gastroenterol 2018; 53:379-389.
47. Smids C, Horjus Talabur Horje C S, Drylewicz J, et al. Intestinal T Cell Profiling in Inflammatory Bowel Disease: Linking T Cell Subsets to Disease Activity and Disease Course. J Crohns Colitis 2018; 12:465-475.
48. Noble A, Durant L, Hoyles L, et al. Deficient Resident Memory T Cell and CD8 T Cell Response to Commensals in Inflammatory Bowel Disease. J Crohns Colitis 2020; 14:525-537.
49. Sun M, He C, Cong Y, et al. Regulatory immune cells in regulation of intestinal inflammatory response to microbiota. Mucosal Immunol 2015; 8:969-978.
50. Ma E H, Poffenberger M C, Wong A H, et al. The role of AMPK in T cell metabolism and function. Curr Opin Immunol 2017; 46:45-52.
51. Arab H H, Al-Shorbagy M Y, Saad M A. Activation of autophagy and suppression of apoptosis by dapagliflozin attenuates experimental inflammatory bowel disease in rats: Targeting AMPK/mTOR, HMGB1/RAGE and Nrf2/HO-1 pathways. Chem Biol Interact 2021; 335:109368.
52. Chassaing B, Srinivasan G, Delgado M A, et al. Fecal lipocalin 2, a sensitive and broadly dynamic non-invasive biomarker for intestinal inflammation. PLoS One 2012; 7:e44328.
53. Gallo L A, Ward M S, Fotheringham A K, et al. Once daily administration of the SGLT2 inhibitor, empagliflozin, attenuates markers of renal fibrosis without improving albuminuria in diabetic db/db mice. Sci Rep 2016; 6:26428.
54. Li C, Zhang J, Xue M, et al. SGLT2 inhibition with empagliflozin attenuates myocardial oxidative stress and fibrosis in diabetic mice heart. Cardiovasc Diabetol 2019; 18:15.
55. Nair A B, Jacob S. A simple practice guide for dose conversion between animals and human. J Basic Clin Pharm 2016; 7:27-31.
56. Rieg T, Masuda T, Gerasimova M, et al. Increase in SGLT1-mediated transport explains renal glucose reabsorption during genetic and pharmacological SGLT2 inhibition in euglycemia. Am J Physiol Renal Physiol 2014; 306:F188-93.
57 Kanada S, Koiwai K, Taniguchi A, et al. Pharmacokinetics, pharmacodynamics, safety and tolerability of 4 weeks' treatment with empagliflozin in Japanese patients with type 2 diabetes mellitus. J Diabetes Investig 2013; 4:613-7.
58. Macha S, Dieterich S, Mattheus M, et al. Pharmacokinetics of empagliflozin, a sodium glucose cotransporter-2 (SGLT2) inhibitor, and metformin following co-administration in healthy volunteers. Int J Clin treatmentPharmacol Ther 2013; 51:132-40.
59. Seman L, Macha S, Nehmiz G, et al. Empagliflozin (B I 10773), a Potent and Selective SGLT2 Inhibitor, Induces Dose-Dependent Glucosuria in Healthy Subjects. Clin Pharmacol Drug Dev 2013; 2:152-61.
62. Heise T, Seman L, Macha S, et al. Safety, tolerability, pharmacokinetics, and pharmacodynamics of multiple rising doses of empagliflozin in patients with type 2 diabetes mellitus. Diabetes Ther 2013; 4:331-45.
63. Friedrich C, Metzmann K, Rose P, et al. A randomized, open-label, crossover study to evaluate the pharmacokinetics of empagliflozin and linagliptin after coadministration in healthy male volunteers. Clin Ther 2013; 35:A33-42.

All publications, patents and patent applications mentioned in this Specification are indicative of the level of skill those skilled in the art to which this invention pertains and are herein incorporated by reference to the same extent as if each individual publication patent, or patent application was specifically and individually indicated to be incorporated by reference.

The invention being thus described, it will be obvious that the same may be varied in many ways. Such variations are not to be regarded as a departure from the spirit and scope of the invention, and all such modification as would be obvious to one skilled in the art are intended to be included within the scope of the following claims.

1. A method of treating a subject with ulcerative colitis (UC), comprising administering a therapeutically effective amount of Empagliflozin (EMPA).

2. A method of treating a subject with Crohn's disease (CD), comprising: administering a therapeutically effective amount of Empagliflozin (EMPA).

3. The method of claim 1, wherein the subject is a human.

4.-8. (canceled)

9. A kit for treating a subject with ulcerative colitis (UC), comprising: Empagliflozin (EMPA), a container, and optionally instructions for the use thereof.

10. A kit for treating a subject with Crohn's disease (CD), comprising: Empagliflozin (EMPA), a container, and optionally instructions for the use thereof.

11. The method of claim 2, wherein the subject is a human.
